(19) United States
(12) Patent Application Publication
(10) Pub. No.: US 2022/0296080 A1
(43) Pub. Date: Sep. 22, 2022

(54) AN IMAGING DEVICE WITH AN INFLATABLE COVER

(71) Applicant: FERTIGO MEDICAL LTD., ZICHRON YA'AKOV (IL)

(72) Inventor: TSAFRIR KOLATT, ZICHRON YA'AKOV (IL)

(21) Appl. No.: 17/636,263

(22) PCT Filed: Aug. 20, 2020
(86) PCT No.: PCT/IL2020/050913

§ 371 Date: Feb. 17, 2022

(30) Foreign Application Priority Data
Aug. 20, 2019 (IL) 268806

(51) Int. Cl. A61B 1/00; A61B 1/05; A61B 1/06; A61B 1/303; A61B 1/015

(52) U.S. Cl. CPC A61B 1/00082; A61B 1/05; A61B 1/0684; A61B 1/303; A61B 1/063; A61B 1/015

(57) ABSTRACT

An imaging device comprising an imaging module and at least one inflatable support element, able to cover and expose the imaging module upon inflation.

### TECHNOLOGICAL FIELD

The present disclosure relates to an imaging device of body regions and specifically imaging of body cavities.

### BACKGROUND ART

References considered to be relevant as background to the presently disclosed subject matter are listed below:
US patent application publication No. 2014180007.
International application publication No. WO2015/151102.
Saab, M A., “Applications of High-Pressure Balloons in the Medical Device Industry,” Medical Device & Diagnostic Industry, September 2000, pp. 86-97.

Acknowledgement of the above references herein is not to be inferred as meaning that these are in any way relevant to the patentability of the presently disclosed subject matter.

### BACKGROUND

The use of camera and specifically miniature cameras is growing in healthcare applications, partly due to the fact that it can provide in-vivo visual exploration inside the human body, without the need for highly invasive surgery.

International application publication No. WO2015/151102 describes a monitoring system comprising: a uterine insert comprising an insert extension, wherein the insert extension comprises at least one sensor; a deployment module engageable with the insert, and configured to allow the insert extension to bend when the module is engaged with the insert; a system control device, and a display operationally coupled to display the data from the system control device.

US patent application publication No. 2014180007 describes an imaging device including an imaging module; a lens associated with the imaging module; and an optically transmissive inflatable member surrounding the lens.

Saab, M A describes in a review article advances in design and fabrication of medical balloons and their medicinal applications.

### GENERAL DESCRIPTION

Imaging of body regions such as body cavities that are not easily accessible, is highly challenging as it requires optimization of a variety of factors including, inter alia, proper delivery and adjustment of an imaging device in the body cavity as well as optimization of imaging conditions as to obtain high quality reliable images.

The quality of images obtained from tissues and specifically tissues within body cavities may be affected by a variety of parameters including technological and physiological ones, that may ultimately obscure and scatter the image scene resulting in blurry images. Such parameters include, inter alia, motion of an imaging device such as a camera or an optic fiber, with respect to the captured scene (tissue), presence of tissue debris, body fluids (such as non-transparent fluid, turbulent fluid or bubbles), mucin, mucus and other bodily elements as well as dirt on the camera lens and/or light source. As appreciated, in-vivo imaging is often affected by natural motion or trembling of the human body that is not only unstoppable (e.g., due to breathing), but also difficult to predict or control. Peristaltic motion, trembling and blood pulsation, may all lead to relative kinetics of a camera and the imaged scene (e.g., tissue). Moreover, obtaining a magnified high resolution and short focal distance image in limited spaces such as a body cavity is even more challenging. The motion timescale and the amplitude dictate the image smearing level which translates to large effects due to the (small) physical dimensions of the field of view to be imaged. While the relative motion may be solved by shortening the exposure time, practically, imaging conditions hinder this solution due to limited illumination power and the numerical aperture values of the camera. The illumination power may be limited for several reasons: to refrain from excess heating by the illumination, to avoid saturation, limited power supply, either momentary or due to prolonged imaging processes and an illumination element distance from the target.

While being challenging, imaging of body cavities is highly important as it may provide information on the tissue's condition by non-invasive means, e.g. without the need for an invasive interference such as surgery. Thus, there is a need for developing imaging device that enable reliable, clear and high-resolution images of body tissue.

The present disclosure is based on the development of an imaging device comprising electrical elements and mechanical elements, packed in a manner that enables delivery into a body cavity and obtaining reliable and accurate images of the body cavity (confined tissue/organ) and any tissue therein. Specifically, the imaging device of the present disclosure comprises an imaging module (being for example a camera or an optical fiber) and at least one support element.

As described herein, when images are recorded by the imaging device, the imaging module is exposed, i.e. not covered/surrounded by a component of the imaging device. In other words, the imaging module can record an image of the tissue directly without having any barrier from the imaging device on the optical axis. It was suggested by the inventors that the use of an exposed imaging module during image acquisition is highly advantageous over a closed/covered imaging module, for example, imaging module that is fully closed/fully surrounded/fully covered during image acquisition, by an element such as a support element. The use of an imaging module that is fully surrounded by a support element forming a cover requires the cover to be transparent and have a similar refractive index as of its surroundings in order to allow transmission of light with minimal scattering or reflection. In such case, if the transparency feature is reduced (for example due to dirt, repeated usage etc.), image quality is negatively affected. Cleaning of such closed elements/covers is highly challenging and may result in the loss of the element integrity and/or clarity.

As detailed herein, the imaging module and optionally at least the lens is exposed, i.e. not covered during imaging.

As also described herein, the support element has a unique ability to undergo reversible changes in the size, volume, width or shape and specifically is configured to undergo inflation (expansion) and/or de-inflation. These changes allow the imaging device to be introduced into the body with the support element in an inflated configuration. As appreciated, an imaging device being introduced with the support element in an inflated configuration has a reduced shape/volume/size and hence enabling the imaging device to be more easily inserted and/or adjusted in place in the body.

During or after the imaging device is being correctly placed within the body, the support element undergoes a change to an expended configuration.

It was suggested by the inventors that once in an expended (inflated) configuration, the support element may stabilize the imaging device within the body cavity, enabling to overcome difficulties associated with imaging of human tissue in small spaces as a result of relative motion of the imaging module and the imaged tissue. It was suggested that both the lateral motion and the motion along the optical axis may be overcome by use of the imaging device described herein. Specifically, it was suggested by the inventors that the imaging device describes herein may “lock” the observed object (e.g., tissue in a body cavity) onto the imaging module, and constitutes an imaging module-object frame that becomes a single “rigid” body with minimal relative motion between the imaging module and the tissue object. This was suggested to provide sufficient solution for the lateral motion. To minimize the relative motion along the imaging module -target axis, it was suggested by the inventors that the support element may be configured to reach a fixed distance with respect to the imaging module specifically lens plane and by that provides a constant focal distance to match the lens characteristics with respect to the captured tissue.

Hence, the combination of an exposed imaging module and a support element that stabilizes the device within the body, provides a free optical axis, during image acquisition between the imaging module and the tissue of interest with no interference of the support element or any other element of the imaging device.

Thus, in accordance with some aspects, the present invention provides an imaging device comprising an imaging module and at least one support element, wherein the support element or any part thereof is configured to undergo inflation in response to a change in pressure.

The “imaging module” as described herein refers to a mechanical, digital, or electronic viewing device, capable of recording, storing, or transmitting visual representation, for example of a tissue, organ or the like. The imaging device is configured to scan a tissue of interest with a detector or electromagnetic beam and specifically in a body cavity of a limited space.

Non-limiting examples of an imaging module include still camera, camcorder, motion picture camera, or any other instrument, equipment, or format configured to capture (record, and the like) an image. The imaging module is configured to capture a still image, sequential images, a video or any combination thereof

In some embodiments, the imaging module is or comprises at least one of a camera or an optical fiber.

In some embodiments, the imaging module is or comprises a camera. In some embodiment, the camera has a lens.

In some embodiments, the imaging module is or comprises an optical fiber. In some embodiment, the optical fiber has a lens.

In some other embodiments, the imaging device comprises an electronic image sensor, for example a charge coupled device (CCD) or a complementary metal-oxide semiconductor (CMOS).

As appreciate the term optical fiber refers to at least a single fiber, possibly a coherent bundle of fibers. In accordance with such embodiments in which the imaging module is or comprises an optical fiber, the camera may be present outside the body and the fiber or fiber bundle is associated with an observing tip carrying photographic information to the electronic image sensor (CCD/CMOS). As appreciated, the electromagnetic signals are collected in the CCD/CMOS. In some embodiments, the electronic image sensor is located remotely to the imaged tissue.

In some other embodiments, the electronic image sensor is located outside the body.

In accordance with some embodiments, the imaging module comprises the observing tip and the electronic image sensor.

As described herein, the imaging device is configured to be introduced into confined body lumen/spaces having a size less than 50 millimeters, less than 10 millimeters and as such the camera within the imaging device should be a miniature camera. Typically, a camera smaller than 3.2mm in diameter may be considered as a miniature camera.

In some embodiments, the imaging module or at least part thereof is covered/surrounded by an element/component of the imaging device such that when inserted into the body and specifically into the body cavity, the imaging module is being exposed (i.e. not covered).

In some embodiments, the imaging module or at least part thereof is covered/surrounded by the support element and during image acquisition, the imaging module is being exposed (i.e. not covered).

As described herein, the imaging module or at least part thereof is exposed during image acquisition. In some embodiments, the imaging module is configured to allow, at least during image acquisition, a direct transmission of light from a captured tissue to the imaging module.

The “support element” at times denoted herein as “inflatable element” or “expandable element” in accordance with the present disclosure, refers to a component of the imaging device that is configured to undergo a change in the shape, area, width, length, area or volume, optionally in response to a change in pressure or in response to a network of springs that expand due to release of its confinement.

The term support element as used herein refers to at least one support element or to a support element having various compartments, each of the compartments is configured to undergo a change as described herein independently.

In some embodiments, the support element is configured to undergo a change in the shape, area, width, length, area or volume of the support element.

In some embodiments, the support element is configured to undergo a change in the shape, area, width, length, area or volume of the support element in response to a change of pressure.

In some embodiments, the support element or at least part of thereof is configured to undergo an expansion (inflation, expansion). The expansion refers to an increase in at least one of shape, area, width, length, area or volume of the support element or any part thereof in response to a change, for example an increase in the pressure, as compared to the shape, area, width, length, area or volume respectively, before the change in pressure.

In some embodiments, the support element or at least part of thereof is configured to undergo a compression (deflation, reduction). The deflation refers to a decrease in in at least one of shape, area, width, length, area or volume of the support element or any part thereof in response to a change, for example a decrease (reduction) in the pressure, as compared to the shape, area, width, length, area or volume, respectively, before the change in pressure.

In some embodiments, the support element is configured to have at least two different configurations.

In some embodiments, the support element is configured to undergo a change between the at least two different configurations.

In some embodiments, the support element or any part thereof is configured to undergo a change between an “unexpanded” (deflated) configuration and “expanded” configuration and vice-versa.

In some embodiments, the support element or any part thereof is configured to undergo a change between the deflated configuration and the expended configuration in repose to a change in pressure, specifically an increase in pressure.

In some other embodiments, when in a deflated configuration, the support element is configured to undergo expansion, for example, in response to pressure, into an expended configuration.

In some embodiments, the support element is inflated along an optical axis.

In some embodiments, the support element is inflated in a perpendicular direction to an optical axis.

As detailed herein, the support element is typically inflated upon insertion of the imaging device into the body and specifically after the insertion of the support element and the imaging module into the body cavity. Hence, the degree of expansion (inflation) may be affected by the specific application to which the device is used, i.e. the body cavity to which the imaging device is introduced/inserted and the tissue to be imaged. In some embodiments, the support element is configured to undergo inflation along an optical axis until it touches the object (e.g., tissue) and then the inflation stops.

Accordingly, and in accordance with some embodiments, when in expended configuration, the support element is configured to act as a fixation means of the imaging device.

In some embodiments, when in expended configuration, the support element is configured to fix the imaging device to a neighboring/surrounding tissue. It was suggested by the inventors that the at least one support element being in an inflated configuration assist in anchoring (fixing) the imaging device in place such that during image acquisition a substantially fixed distance of the imaging device, specifically the imaging module from the imaged target (e.g., tissue) is obtained, reducing lateral and axial relative motion of the imaged target with respect to the imaging device.

The support element when in an inflated configuration may, in some embodiments, provides a barrier maintaining a “clear” free space of medium between the imaging module as well as other components of the imaging device and the imaged target.

In some embodiments, the support element comprises at least one nozzle. As shown in the Figures below, the support element may comprise at least one nozzle, with or without a valve.

In accordance with some embodiments that may be considered as some aspects of the invention, when being in a deflated (unexpended) configuration, the support element covers/surrounds/enclose the imaging module, optionally at least the lens. In accordance with such embodiments, the support element is configured to surround (cover) the imaging module or any part thereof such that it protects the imaging module. In accordance with such embodiments, the support element is in an deflated configuration surrounding the imaging module or at least a part thereof and only during or after the imaging device is being correctly placed within the body, the support element undergo a change to an expended configuration resulting in exposure (uncover) of the imaging module or at least part thereof. It was suggested that introducing the imaging device with the support element covering the imaging module protects the imaging module during insertion.

Regardless of the arrangement of the imaging module and the support element, it should be noted that when in an inflated (expanded) configuration, the support element does not cover the imaging module and optionally at least the lens.

The support element in accordance with the present disclosure when being in an inflated configuration within the body cavity may enable flow of gas and/or fluids to the imaged tissue or through the imaged tissue (for example a blood vessel or heart) as well as the surroundings as well as drainage or gas/fluid supply from and to the tissue.

The support element forming part of the imaging device is introduced into the body and hence may be composed from any material that is suitable for being introduced into the body.

In some embodiments, the support element comprises a biocompatible material. In some embodiments, the support element comprises a biomaterial.

In some embodiments, the support element comprises a disposable material.

In some embodiments, the support element is or comprise a material that can be stretched or expand.

In some embodiments, the support element is or comprise an elastomeric material.

In some embodiments, the support element is or comprises a flexible material.

In some embodiments, the support element is or comprises an elastic material. As appreciated, an elastic material encompasses any solid material that has a tendency of stretching and returning to the original shape or size after forces are released.

In some embodiments, the support element is or comprise different materials having different flexibility and elasticity characterization.

Examples of an elastic material include polyethylene, nylon, latex, or silicone.

In some other embodiments, the support element is or comprises a non-elastic material. Examples of a non-elastic material include polyethylene terephthalate (PET).

In some other embodiments, the support element is or comprises a semi-elastic material. Examples of a semi-elastic material include nylon.

In some other embodiments, the support element comprises a combination of elastic and non-elastic materials. In some other embodiments, the support element comprises a combination of elastic and rigid materials. It should be noted that when referring to a support element that is configured to undergo inflation and/or deflation, the part of the support element that is elastic/flexible undergoes the change.

In some embodiments the support element comprises of a membrane.

In some embodiments, the support element is or comprises a balloon.

The term balloon as used herein denotes a member that can undergo at least one of inflation and/or deflation.

The support element, for example being a balloon may be comprised of materials of several different flexibility and elasticity characteristics (usually referred to as being “complaint”, “non-compliant” or “semi-compliant”). In some embodiments, the support element is or comprises a compliant material, a semi-compliant material, a non-compliant material or any combination thereof. The balloon may be comprised of a combination of rigid and flexible elements. The balloon may have maximal expanding volume, form, radius, etc. and may comprise of several compartments.

In some embodiments, the support element has a pre-design shape when in inflated configuration. In some embodiments, the pre-design shape is achieved by use of a homogeneous elastic material to form the specific three-dimensional structure of the support element when in inflated configuration.

As appreciated in order to obtain high resolution images of body cavities, an illumination source is often required. In some embodiments, the imaging device comprises an illumination element.

The term “illuminating element” or “illumination means” as used herein refers to a light source being configured to allow enough reflecting electromagnetic radiation for the acquisition of at least one image of a human tissue. It should be noted that the term illumination means as used herein encompasses filters, prisms, mirrors, polarizers, spectral acquisition elements (e.g., grid, grate, etc.).

In some embodiments, the illumination means comprise at least one of a light-emitting diode (LED), a laser, or an optical fiber carried signal.

In some embodiments, the illumination means comprise at least one LED.

It was suggested that the support element enables to position the illumination means in relative angles and distances with respect to the imaged tissue and to the imaging module hence assisting in obtaining a high-resolution image.

In some embodiments, the imaging module and the support element are directly associated/connected, i.e., have at least one point/location of physical contact. In some embodiments, at least two of the imaging module, the support element and the illumination means are directly associated, i.e., have at least one point/location of physical contact.

In some embodiments, the imaging module and the support element are indirectly associated, for example, both are connected to a body element acting as a mediator between the imaging module and the support element. In some embodiments, the imaging module, the support element and the illumination means are indirectly associated, for example, both are connected to a body element acting as a mediator between imaging module, the support element and the illumination means.

The portion of the imaging device defined by the imaging module, the support element and optionally the illumination means and optionally the mediator commencing at least two of the imaging module, the support element and the illumination means is at times referred herein as the insertable portion or the imaging portion. As described herein, the insertable portion is configured to be inserted into a body cavity and in accordance with some embodiments, to be positioned in the body and specifically the body cavity for extended/long time period.

The imaging device may be packed as a capsule and may be administrated per se into the body for example by swallowing.

In accordance with some embodiments, when being in a deflated (unexpended) configuration, the support element and the imaging module and optionally the illumination elements are packed. In accordance with such embodiments, the support element is in an inflated configuration and only during or after the imaging device is being correctly placed within the body, the support element undergo a change to an expended configuration resulting in exposure (uncover) of the imaging module or at least part thereof.

Alternatively, and in accordance with some embodiments, the imaging device should be inserted into the body and/or guided within the body and into the body cavity.

In some embodiments that may be considered as aspects of the invention, the imaging device comprise a delivery member.

In some embodiments, the imaging device comprises an imaging module, a support element and a delivery member.

The delivery member as used herein refers to a shaft, a handle or a catheter that is configured to allow introduction/insertion of the imaging device into the body and adjustment, such as spatial adjustment of the imaging device within the body cavity to be imaged.

In some embodiments, the delivery member may be a disposable element or a recyclable element. In some embodiments, the delivery member may be flexible.

In accordance with some embodiments, the delivery member is configured to allow distal application of the imaging device, for example the camera.

In some embodiments, the association between the imaging module, the support element and the delivery member may be a direct association or an indirect association.

In some embodiments, the imaging module, the support element optionally the illumination means and the delivery member are directly associated, i.e., have at least one point/location of physical contact between two of the above-mentioned components.

In some embodiments, imaging module, the support element, optionally the illumination means and the delivery member are indirectly associated, for example, at least two are connected to an interface element acting as a mediator between the imaging module, the support element and the delivery member. The interface/mediator element may be located at any part of the imaging device. In accordance with some embodiments, the interface element is located at a proximal end of the support element.

In accordance with some embodiments, the delivery member is configured to undergo association and/or dissociate from any part of the imaging device. In accordance with some embodiments, the delivery member is configured to associate and/or dissociate from the imaging portion. In accordance with some embodiments, the delivery member is configured to undergo dissociation from the imaging portion.

It should be noted that the delivery member undergo dissociation from the imaging portion after the imaging device and specifically the imaging portion is placed within the body part to be imaged, for example, within the body cavity to be imaged and the tissue within the body cavity.

It should be further noted that the association and dissociation of the delivery means from a part of the imaging device and specifically the imaging portion can be done by any known connection method allowing reversible associational and dissociation. In some examples the delivery element is connected to a part of the imaging device and specifically the imaging portion by a plug-in element present at each one of the parts that undergo dissociation so that the two parts are detachable and in some example can be locked together.

Hence and in accordance with some embodiments, after insertion of the imaging device into the body, the delivery member and/or the interface member may be configured to be removably coupled. In some embodiments, after insertion of the imaging device into the body, the delivery member and/or the interface member may be configured to be removably coupled. In some embodiments, after insertion of the imaging device into the body, the delivery member and/or the interface member may be configured to be removably coupled and the imaging portion is allowed to be placed in the body cavity for a time period as described herein.

The imaging device may be packed in a variety of arrangement, depending for example on the mode of administration. As appreciated, the mere anatomy of the explored region (tissue/organ) plays an important role in the design of the imaging device arrangement. The association between the imaging module, the illumination element and the imaged tissue should satisfy various conditions in order to be able to record images of high quality. Such conditions include, for example, lack of obstructions along the lines between the illumination elements and the recorded tissue as well as the imaging module camera and the recorded tissue (line of sight), avoidance of camera saturation due to direct or reflected light, flexibility in the triangle angles (e.g., illumination angle with respect to the captured tissue), the captured tissue shape and color, etc.

As shown in the Figures below, the imaging device may have a variety of arrangements, structures and sizes depending, inter alia, on the size and the structure of the body cavity to be imaged and/or how it is being introduced into the body.

FIGS. 1A to 1H show exemplary embodiments of imaging device showing the support element in an inflated configuration. In some embodiments, the support element in an inflated configuration has a concave structure. In some embodiments, the support element in an inflated configuration has a balloon structure. A representative embodiment of the imaging device with and without a delivery member is shown in FIGS. 1A and 1B, respectively which may apply to all imaging devices encompassed by the present disclosure. In other words, while the figures below show exemplary embodiments of an imaging device comprising a delivery member, the present disclosure also encompasses such imaging device lacking a delivery member. The part of the imaging device shown in FIG. 1B as an exemplary representation of the imaging device without the delivery means is at times denoted as the imaging portion.

It should be further noted that the support element in exemplary embodiments being a balloon with different shapes when in inflated configuration, is in a folded or pleated position about itself or about the delivery member in the deflated configuration.

In some embodiments, the imaging device may comprise a pressure system.

The pressure system according with the present disclosure provides fluid and/or gas and/or air to inflate and/or deflate the at least one support element or any part thereof. In some embodiments, the pressure system comprises a pump and/or a tube network and/or a release valve. The valves may be located at the inlet of each support element, at the pump outlet or in between. Valves can be mechanically or electronically controlled, either spontaneously (e.g., beyond some pressure value) or by the user trigger. In some embodiments, inflating of the support element may be controlled by at least one of the following: the pump pressure and pressure build-up process, the pump total power, the gas/liquid maximal/minimal pumped volume or the pump activation/de-activation mechanisms, e.g., sensors attached to the support element surface to determine the pump operational mode.

As appreciated, for the operation of the imaging device and specifically for the change in the support element, the imaging device may comprise of at least one mechanical element that allows, controls or mediates mechanical forces, tension, compression, or other bias within the imaging device.

In some other embodiments, the support element comprises at least one mechanical element. In some embodiments, the delivery member comprises at least one mechanical element. In some embodiments, a mechanical connection may exist between at least one mechanical element present on the support element and at least one mechanical element on the delivery member. In some embodiments, the mechanical connection is at the interface member.

It was suggested that the mechanical connection (either directly or via the interface member) may allow for mechanical forces, tension, compression, or other bias to be to be passed from the delivery member to at least one component of the imaging device, including, inter alia, the support element. It was further suggested that the mechanical connection (either directly or via the interface member) may allow for mechanical forces, tension, compression, or other bias to be to be passed from at least one component of the imaging device, including, inter alia, the support element to the delivery member. In some embodiments, the mechanical connection allows fluid and/or gas to be pressurized to inflate the inflatable element. As described herein, the support element may be selectively inflated and deflated with proper fluid, such as air or saline, or gas etc.

In some embodiments, at least one of the imaging module, the support element, the delivery member and the interface member may include one or more engagement features. The engagement features when coupled together, prevent inadvertent decoupling of components of the imaging device.

In some embodiments, the imaging device may comprise electrically conductive elements. The electrically conductive elements may be configured for providing electrical power to one or more electrical elements, such as the imaging module and/or illumination elements. In some embodiments, the power to the imaging module (camera) and/or power to the illumination means (light source) may be located in the imaging device, in the delivery member or in the interface member.

In some embodiments, the power to the imaging module, the power to illumination means and the mechanical force and fluid and/or gas and/or air pressure to the support element, may be present in the imaging device, in the corresponding delivery member, or in an interface member.

The imaging device may comprise in accordance with some embodiments at least one control unit configured to control operation of any part of the device at least the imaging module and/or the support element. The control unit is, in accordance with some embodiments, configured to switch the support element between its deflated and inflated positions (configurations). Switching the support element into the inflated configuration can depend on various factors in order to ensure inflation only when the imaging device and specifically the imaging portion is located in place within the body cavity.

In some embodiments, the device may comprise at least one sensor associated with the control unit. Reading from the sensor can be used to provide indication on proper operation of the device.

According to some embodiments, the device may further comprise at least one display module associated with the control unit and configured to display information obtained from the imaging module. For example, the display module may be configured to display information obtained by the imaging module, e.g. images of the tissue being imaged. The display module can be of any suitable type, for example a visual display (e.g. an electronic display panel, an LED display panel, a screen, etc.).

The imaging device described herein may be used for imaging of a variety of tissues. As appreciated, specific adaption of the illumination elements, and the optics (field of view, angle of view, magnification, focal depth and distance, etc.), is required depending on the tissue to be imaged. The selected solution should also take into account the surrounding environment that may affect, for example, the light reflections, the image medium and the image target. This should also dependent on accompanying image processing equipment (software and hardware) for optimal results in the context of the specific application it addresses.

In accordance with some aspects, the present disclosure provides a method of imaging a subject, the method comprises applying/inserting/introducing an imaging device to a subject in need thereof and monitoring a signal from the subject. Specifically, the method comprises imaging at least one tissue within at least one body cavity.

In accordance with some embodiments, the method comprises applying/inserting/introducing an imaging device, the imaging device comprises an imaging module and a support element. In accordance with some embodiments, the method comprises applying an imaging device, the imaging device comprises an imaging module, a support element and an illumination element. In accordance with some embodiments, the method comprises applying an imaging device, the imaging device comprises at least an imaging module, a support element, an illumination element and a delivery member.

The term “monitoring” as used herein is meant to encompass the quantitative and/or qualitative detection and observation of a signal originating from the region been imaged by the imaging device, optionally over a period of time. It should be noted that monitoring a signal encompasses collecting data possibly in a form of a signal or an image from a subject including any region of the subject body. The region/tissue in accordance with the present disclosure may be located within a body cavity.

In some embodiments, the method comprises monitoring a signal or an image from at least one body cavity as described herein.

In some embodiments, the method comprises recording at least one image from at least one body cavity as described herein (tissue/organ).

As appreciated, monitoring a signal or an image from a tissue may provide valuable information in real time on the condition of the imaged tissue including, inter alia, information on a pathological condition, maturation of cells, status of blood vessels etc. The information may be used for diagnosis purposes.

In some embodiments, the imaging device may be for use in diagnosing a condition or a disease in a subject. Hence, in accordance with some embodiments that may be considered as aspects of the invention, there is provided a method for diagnosing a condition or disease in a subject in need thereof, the method comprises inserting an imaging device to the region to be diagnosed and monitoring a signal, thereby diagnosing the condition or disease in the subject.

The term “diagnosing a condition or disease” is meant to encompass any process of investigating, identifying, recognizing, assessing a condition, disease or disorder of the mammalian body, including all organs, tissues and structures in the body.

The term body cavity as used herein refers a space or compartment, or potential space in the body that accommodate organs and other structures; as well as cavities as potential spaces containing fluid.

In some embodiments, the body cavity is ventral body cavity or dorsal body cavity.

In some embodiments, the body cavity is the dorsal body cavity. In some embodiments, body cavity is the dorsal body cavity comprising the cranial cavity. The cranial cavity is enclosed by the skull.

In some embodiments, the cranial cavity comprises the brain.

In some embodiments, body cavity is the dorsal body cavity comprising the vertebral canal.

In some embodiments, the vertebral canal comprises the spinal cord.

In some embodiments, the body cavity is the ventral body cavity.

In some embodiments, body cavity is the ventral body cavity comprising the thoracic cavity. The thoracic cavity is enclosed by the ribcage.

In some embodiments, the thoracic cavity comprises the lungs and heart.

In some embodiments, body cavity is the ventral body cavity comprising the abdominopelvic cavity.

In some embodiments, the abdominopelvic cavity comprises the abdominal cavity and the pelvic cavity.

In some embodiments, the abdominal cavity comprises the digestive organs, spleen and kidneys.

In some embodiments, the pelvic cavity comprises the bladder and reproductive organs.

In some embodiments, the body cavity is at least one of heart, lungs, liver, kidney, stomach, intestines, thymus, pancreas, skin, bone, bone marrow, vascular system, lymph, lymph node, uterus, bladder, fallopian tubes, ovaries, a joint.

In some embodiments, the body cavity is a joint. In some embodiments, the joint is at least one of joints of hand, elbow joints, wrist joints, axillary articulations, sternoclavicular joints, vertebral articulations, temporomandibular joints, sacroiliac joints, hip joints, knee joints or articulations of foot.

In some embodiments, the body cavity a shoulder.

In some embodiments, the body cavity is a reproductive organ.

In some embodiments, the body cavity is a uterus.

In some embodiments, the body cavity is a bladder.

The condition as used herein may encompasses any physiological process.

The condition may not necessarily be a pathological condition.

In some embodiments, the condition is associated with the reproductive system.

In some other embodiments, the method comprises monitoring uterus of a female.

In some embodiments, the condition or disorder is related with the uterus (endometrium) status. For example, the standardized cycle day to which the uterus resembles the most. The latter can be inferred, e.g., by the density, shape, distribution and size of tissue elements such as gland openings (“pores”) or blood vessels. Hence, the condition that can be diagnosed for a tissue as described herein relates to assessment of blood vessels including density, shape, distribution and size.

In some embodiments, the condition relates with the gastrointestinal system.

In some embodiments, the condition relates with the cardiovascular system.

In some embodiments, the disease or disorder is related to oncology, neurology, psychiatry, cardiology, vascular, infection and inflammation diseases and disorders.

In accordance with some embodiments, the methods comprise inserting the imaging device to the body such that the support element is in a deflated configuration. It was suggested by the inventors that the imaging device is introduced into the body with the support element in a deflated configuration to allow effective application (introduction) of the device into the body and adjustment in a body cavity to be imaged.

In accordance with some other embodiments, the methods comprise inserting the imaging device to the body such that the support element is in a deflated configuration and covering the imaging module. It was suggested by the inventors that the imaging device is introduced into the body with the support element in a deflated configuration covering the imaging module may protect the imaging module during insertion.

The methods of the present disclosure comprise a step of allowing the support element to undergo a change. In some embodiments, the method comprises allowing the support element to undergo a change in at least the shape, area, width, length, area or volume.

In some embodiments, the method comprises allowing the support element to undergo a change in at least the shape, area, width, length, area or volume of the support element in response to a change of pressure.

In some embodiments, the method comprises applying pressure in the imaging device to allow the support element to undergo an expansion (inflation) of at least part thereof.

In some embodiments, the method comprises applying pressure in the imaging device to allow the support element to undergo a change from an inflated to an expended configuration.

In accordance with some embodiments that can be considered as aspects of the present invention, the methods comprise the steps of applying an imaging device comprising an imaging module and a support element to a subject, applying pressure and monitoring a signal.

In accordance with some embodiments, applying pressure is done concomitantly to the introduction of the imaging device into the body.

In accordance with some embodiments, application of pressure is done, when the imaging device is in the body and specifically within the body cavity to be imaged.

In accordance with some embodiments applying pressure to the support element takes place during image acquisition.

In some embodiments, the method comprises at least one cycle of inflation and deflation of the support element. In some embodiments, the method comprises at least one cycle of applying pressure (increase pressure) thereby allowing the support element to undergo inflation and reducing pressure to thereby allowing the support element to undergo deflation. The method comprises in accordance with some embodiments, repeated cycles of increase and decrease of pressure.

The degree of the inflation and thereby the degree at which the support element is being inflated depends, among others, on the structure and materials of the support element and/or the required interaction between the element and the tissue as well as the organ anatomy.

In some embodiments, the method comprises inflating the support element along the longitudinal optical axis until it is in contact with the tissue. In some embodiments, the method comprises inflating the support element perpendicular to the longitudinal optical axis until it is in contact with the tissue.

In some embodiments, the method comprises inflating the support element along the longitudinal optical axis, thereby “pushes” the tissue in order to reach a certain predetermined distance (e.g., focal length) and/or alternatively to reach a specific distance from the object (e.g. the tissue) and to expand laterally thereafter so that the support element, for example a balloon stretches the tissue or makes it more planar. Hence, the support element may for example, push away collapsed cavities enabling their imaging.

In some embodiments, the method comprises inflating the support element by use of a pump and a release valve(s). In some embodiments, the method comprises inflate and/or deflate each one of the support elements in parallel. In some embodiments, the method comprises inflate and/or deflate each one of the support elements or its' compartment separately at different times.

In some embodiments, the method comprises inflating at least one support element or any part (i.e. inflatable compartment) thereof to allow fixation of the imaging module-tissue. In some embodiments, the method comprises inflating at least one support element or any part (i.e. inflatable compartment) thereof to provide support for the imaging module.

In some embodiments, the method comprises inflating different inflatable parts of the support element using different pressure. It was suggested that inflating different inflatable parts of the support element using different pressure levels may assist in positing the imaging module in specific location in the body cavity, for example relative to the tissue to be captured.

In some embodiments, the method comprises inflating different inflatable parts of the support element at different times.

In some embodiments, the methods comprise inflating the support element with gas.

In some embodiments, the methods comprise inflating the support element with liquid.

In some embodiments, the method comprises inflating different inflatable parts of the support element with gas and/or liquid. In some embodiments, the method comprises inflating at least part of the support element with gas and other part of the support element with liquid.

Hence, in accordance with the methods described herein, the imaging device is introduced into the body having the support element in a deflated configuration, optionally covering the imaging module or at least part thereof, for example the lens and once being located in the body and specifically the body cavity to be imaged and/or in proximity to the tissue to be imaged, the support element undergo a change from a deflated configuration to an expended configuration, optionally exposing the imaging module and at least the lens.

It should be noted that regardless of the arrangement of the imaging device and as described herein, the imaging module or at least part thereof is exposed during imaging acquisition. It was suggested by the inventors that the expansion of the support element is sufficient to uncover the imaging module such that a direct contact, e.g. free optical axis exists between the imaging module and the tissue to be imaged.

In some embodiments, the imaging module is in direct contact with a tissue to be imaged during image acquisition. The term “direct contact” as used herein indicates that the optical axis between the imaging module and the captured tissue is free from any element of the imaging device, e.g. the support element or any part thereof.

Hence, at times in which the imaging module is exposed and is in direct contact with the tissue to be imaged (captured, recorded), a transmission of light (rays of light) is directed from the imaged organ/tissue to the imaging module. As noted above, it should be appreciated that the direct contact is achieved by the unique features of the imaging device and specifically the support element that does not surround the imaging module at least during image acquisition. This allows light to enter and/or the illumination means, and thus, images from the recorded organ/tissue are captured without substantial interference to the quality of the imaging. In addition, as the support element does not surround the imaging module it enables direct transmission of light (rays of light) from the imaged organ/tissue to the imaging module.

The imaging device may be used for a single time examination (static diagnosis) or it be positioned for a defined time period (dynamic diagnosis) within the organ/tissue to be recorded to obtain multiple images.

The imaging device may be positioned/inserted into the human body for prolonged periods of time and may become active (e.g. acquiring images) at specific instances, upon demand or in accordance with a pre-determined monitoring schedule. In some embodiments, the imaging device and specifically the imaging portion may be positioned within the human body for at least an hour, at least a day, at least a week. In some embodiments, the imaging device and specifically the imaging portion is configured for continuous monitoring of an organ in the human body. In some embodiments, the imaging device is inserted into a human uterus. As appreciated, the human uterus has a structure of a collapsed lumen and in order to be able to capture images of the uterus, the uterus cavity needs to expand.

In some embodiments, after insertion of the imaging device into the body cavity, the methods compress dissociation of at least the delivery member. In some embodiments, the method comprises, after insertion of the imaging device and specifically the imaging portion into the body cavity, removing the delivery member and/or the interface member. In some embodiments, the methods comprise after insertion of the imaging device and specifically the imaging portion into the body cavity, dissociating/removing the delivery member and/or the interface member from the imaging portion. In some embodiments, the methods comprise after insertion of the imaging device and specifically the imaging portion into the body cavity, dissociating/removing the delivery member and/or the interface member from the imaging portion to allow the imaging portion to be placed within the body cavity for a prolong time period.

The methods of the present disclosure also encompass engaging and removing a protective cover from the imaging device, by for example inflation/deflation of the at least one support element. However, in specific embodiments it may also involve strings, springs and other system ingredients that limit the expansion, resist it, bring back the cover into its idle position and assist in placing it correctly. Such ingredients may also bring the deflated support element back to its compact configuration, folded, pleated, wrapped or otherwise stored in close proximity to the imaging module.

Some of the characteristics of device and methods as discussed above are also applicable for other imaging techniques, for example, for Real Time Confocal Laser Endomicroscopy that is mostly used inside the human body but also for in-vivo diagnosis outside the human body .

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

Disclosed and described, it is to be understood that this invention is not limited to the particular examples, methods steps, and device disclosed herein as such methods steps and device may vary somewhat. It is also to be understood that the terminology used herein is used for the purpose of describing particular embodiments only and not intended to be limiting since the scope of the present invention will be limited only by the appended claims and equivalents thereof.

It should be noted that in the text herein, when referring to the imaging device it is to be understood as also referring to the methods disclosed herein. Thus, whenever providing a feature with reference to the imaging device, it is to be understood as defining the same feature with respect to the methods, mutatis mutandis.

It must be noted that, as used in this specification and the appended claims, the singular forms “a”, “an” and “the” include plural referents unless the content clearly dictates otherwise. “a” may be considered as “at least one”.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A to 1H show exemplary embodiments of the imaging device.

FIGS. 2A and 2B show various exemplary configurations of an imaging device.

FIG. 3A to 3C show various exemplary configurations of an imaging device.

FIG. 4 shows an imaging device having a ring-shaped string/spring.

FIG. 5 shows an imaging device having a protective cover according with some embodiments.

FIGS. 6A and 6B show various exemplary configurations of an imaging device.

FIG. 7 shows an exemplary representation of an imaging device according with some embodiments.

FIGS. 8A to 8C show representative examples of the pressure system.

FIG. 9 shows an exemplary embodiment of an imaging device.

### DETAILED DESCRIPTION OF EMBODIMENTS

The invention will now be further described with reference to the exemplary embodiments depicted in the drawings. These exemplary embodiments are meant to illustrate the imaging device of this disclosure but not intended to be limiting in any way. In other words, the scope of this disclosure applies to the full contents of the above disclosure and is not limited in any way to these exemplary embodiments.

The imaging device can have various configurations, such as those illustrated and described herein. It should be noted that features of one figure and description can be combined with any other feature of any other figure and embodiment.

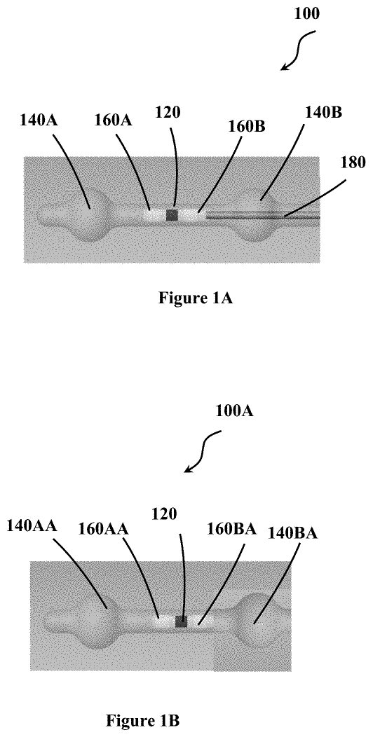

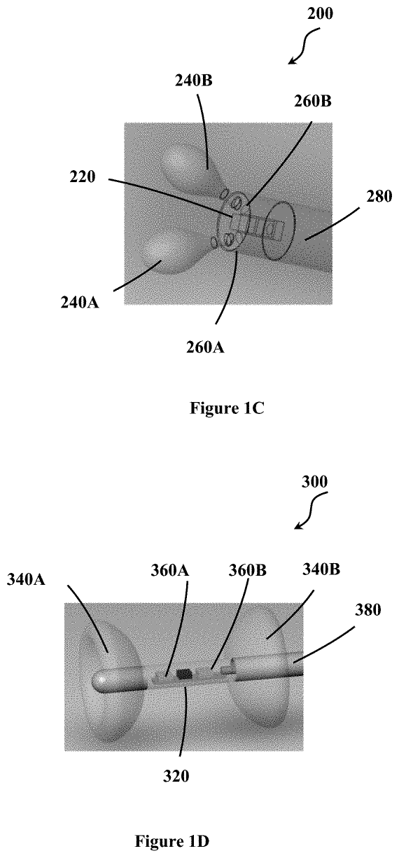

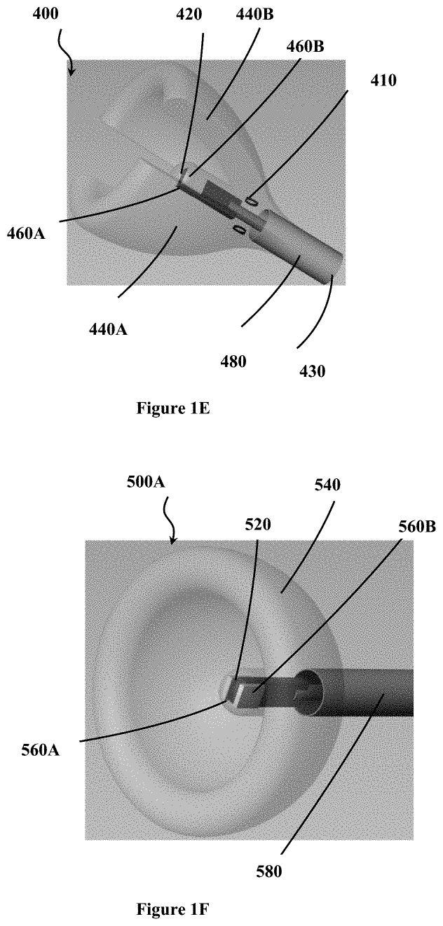

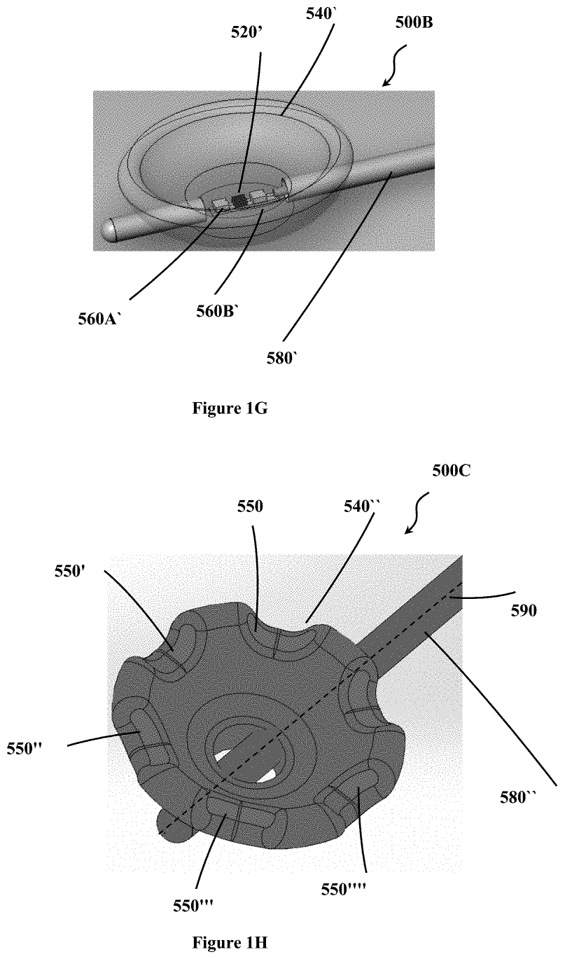

Reference is now made to FIGS. 1A to 1H showing exemplary embodiments of the imaging device according with the present disclosure with the support element being inflated (expended). As described herein, the inflation typically takes place after and/or during the imaging device has been inserted into the body cavity and at least during image acquisition. Hence, the exemplary embodiments shown in FIGS. 1A to 111 may be considered as the imaging device configuration when in the body specifically in the body cavity and at least during image acquisition.

FIGS. 1A shows an exemplary imaging device 100 having an imaging module 120, two support elements 140A and 140B, two illumination means 160A and 160B and a delivery member 180.

Each one of the support elements has an ellipsoid shape in an inflated configuration with an ellipse cross section perpendicular to the plane defined by the imaging module and the illumination means. The two support elements may have spherical shape (not shown). FIG. 1B is an exemplary imaging device 100A which as in FIG. 1A has an imaging module 120, two support elements 140A and 140B and two illumination means 160A and 160B but does not comprise a delivery member. As detailed herein, the exemplary representation provided in FIG. 1B is at times denoted as the imaging portion.

FIG. 1C shows exemplary imaging device 200 having an imaging module 220, two support elements 240A and 240B and two illumination means 260A and 260B and a delivery member 280. Each one of the support elements has a “balloon” shape in an inflated configuration

FIG. 1D shows exemplary imaging device 300 having an imaging module 320, two support elements 340A and 340B and two illumination means 360A and 360B and a delivery member 380. Each one of the support elements in an inflated configuration has a concave structure facing away from the imaging module.

FIG. 1E shows exemplary imaging device 400, having an imaging module 420, two support elements 440A and 440B and two illumination means 460A and 460B and a delivery member 480. Each one of the support elements in an inflated configuration has a concave structure facing towards the imaging module.

The imaging device 400 in accordance with some embodiments, has fluid inlet 410 and a wire assembly 430 for power supply and data transfer.

While each one of the two support elements in FIGS. 1A to 1E have an identical shape, the present disclosure encompasses also two or more different support elements, each having a different shape and/or size in the imaging device as well as when having different shape can be inflated to a different degree.

FIGS. 1F to 1H show exemplary imaging device 500A, 500B and 500C having a support elements 540, 540′ and 540″, respectively having in an inflated configuration a concave structure housing within the imaging module 520 (FIG. 1F) and 520′ (FIG. 1G) and two illumination means 560A and 560B (FIG. 1F) and 560A′ and 560B′ (FIG. 1G). The imaging device in 1F to 1H comprises a delivery member 580, 580′ and 580″, respectively.

The position of the imaging module and as such the imaged tissue with respect to the insertion direction (herein axial axis) of the imaging device depends on specific organ/tissue to be imaged.

The imaging device 500C (FIG. 111) comprises a delivery member 580 that is orientated in a manner that allows insertion of the device into the body via an axis 590. In the description herein, the term axial is used to define a direction parallel to axis 590. The imaging device 500B and 500C face an axis being perpendicular to the axial axis. The support element of 500A and 500B has a continuous concave structure, wherein in imaging device 500C, the support element has grooves (cut-out), 550, 550′, 550″, 550′″.

It was suggested by the inventors that a support element as shown in FIGS. 1G and 1H may be configured to fit and stick onto the captured tissue/organ such that it substantially prevents flow of debris and/or fluids from interfering the line of sight between the imaging module and specifically imaging means and the recorded tissue.

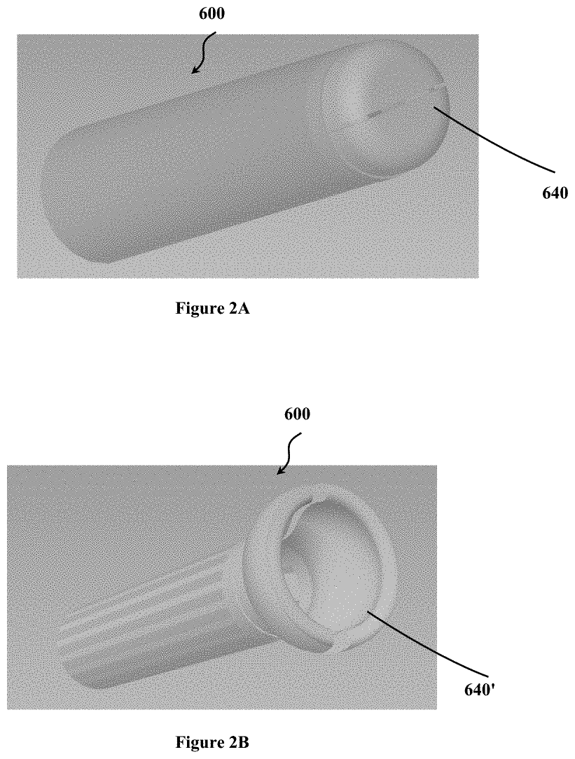

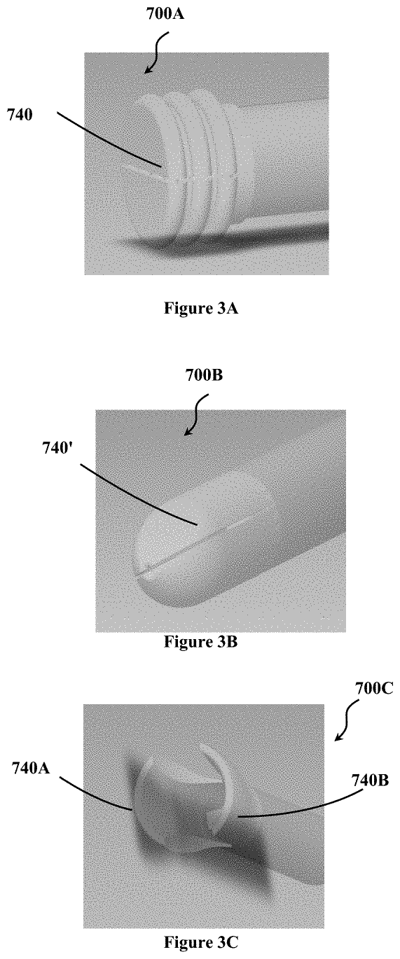

FIGS. 2 and 3 show representative configurations of the support element, in deflated and inflated configurations. In these specific configurations, the support element when in deflated configuration covers the imaging module.

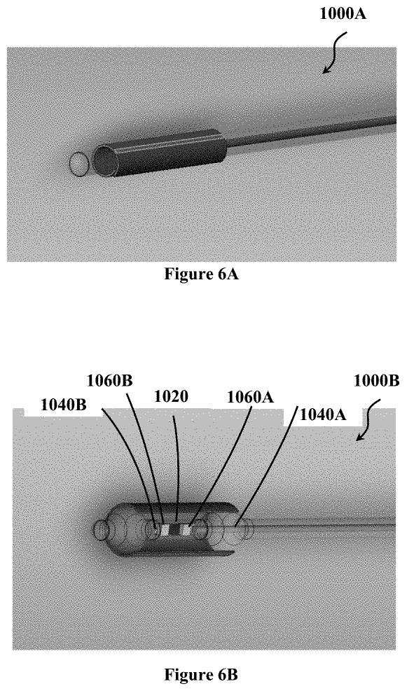

FIGS. 2A and 2B show various configuration of an imaging device 600 according with some embodiments. FIG. 2A, shows the imaging device 600 comprises the support element 640 is in a packed (deflated) orientation surrounding the imaging module (not shown). In such configuration, the support element is a protective cover for the imaging module and/or the illumination means. FIG. 6B shows imaging device 600 having a different configuration of the support element 640′ being inflated (expanded) into a concave structure, exposing the imaging module (not shown). As described herein, these two configurations may be exemplary sequence of operation of the imaging device 600 such that in a first step, seen in FIG. 2A, the imaging device comprises the support element 640 is in a packed (deflated) orientation surrounding the imaging module. FIG. 2B shows the subsequent step occurring in vivo in which, prior to image acquisition, the support element 640′ is inflated (expanded) into a concave structure, exposing the imaging module (not shown).

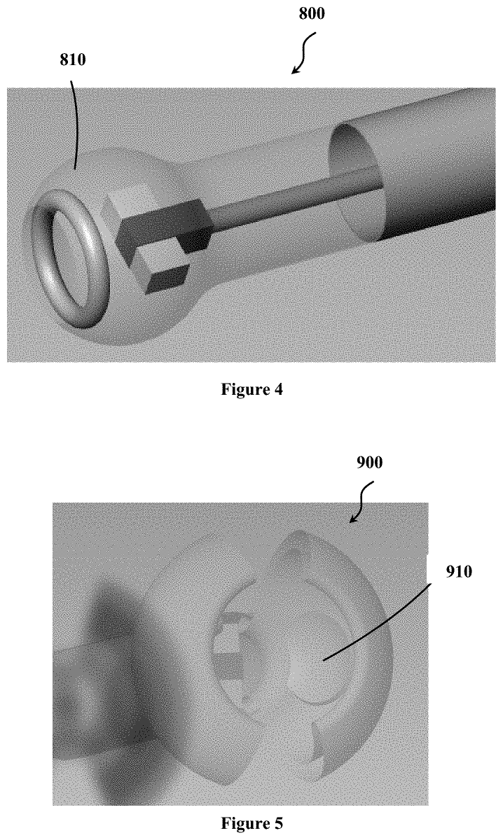

FIG. 3A to 3C show a representative embodiment of an imaging device 700A, 700B and 700C, respectively. The support element 740 is shown in FIG. 3A to have a closed folded configuration configured as a protective cover. FIG. 3B shows a closed stretched configuration of the support element 740′. FIG. 4C shows a differed configuration of the support element being inflated (expanded) into a concave structure comprising 740A and 740B. These three configurations may be exemplary sequence of operation of the imaging device 700 such that in a first step, seen in FIG. 3A, the imaging device comprises the support element 740 is in a packed (deflated) folded orientation surrounding the imaging module. The support element is stretched to a configuration as shown in FIG. 3B. The imaging device/system shown in FIG. 3A or in FIG. 3B is then inserted into the body. It should be noted that the stretching from FIG. 3A to FIG. 3B may take place in vivo. FIG. 3C shows a subsequent step occurring in vivo in which, prior to image acquisition, the support element 740A and 740B (shown in inflated configuration) is inflated (expanded) into two concave structure, exposing the imaging module (not shown). An optional circular string/spring at the support element opening (not shown) may prevent it from opening before the folded structure (harmonica-like) is fully stretched (FIG. 3B). In alternative embodiments, the design of the support element is configured to allow opening of the concave structure only after the support element is fully stretched (FIG. 3C) and may also be used to remove unwanted objects from the line of sight.

FIG. 4 shows an imaging device 800 having a ring-shaped string/spring 810 being stretched under the inflated support element 810 pressure and closes the opening to protect the camera when the support element is in its deflated configuration.

FIG. 5 shows an imaging device 900 having a protective cover 910 being placed in front of the camera as a shield to protect it while not in use. The inflated support element uncovers the lens by drawing 910 aside. When deflated, the cover 910 is placed back to its protective position in front of the camera, using a string, spring or other restraining mechanisms (rails, etc.)

FIGS. 6A and 6B show various configuration of an imaging device 1000 according with some embodiments. FIG. 6A, shows the imaging device packed. FIG. 6B shows imaging device 1000 having an exposed configuration of the imaging device comprising an imaging module 1020, two support elements 1040A and 1040B and two illumination means 1060A and 1060B as well as a delivery member 1080.

As described herein, these two configurations may be exemplary sequence of operation of the imaging device 1000 such that in a first step, seen in FIG. 6A, the imaging device is in a packed orientation. The imaging device is inserted into the body. FIG. 6B shows the subsequent step occurring in vivo in which, prior to image acquisition, the support element 1040A and 1040B are inflated (expanded) and pushing the packing element such that the imaging module 1020 is exposed.

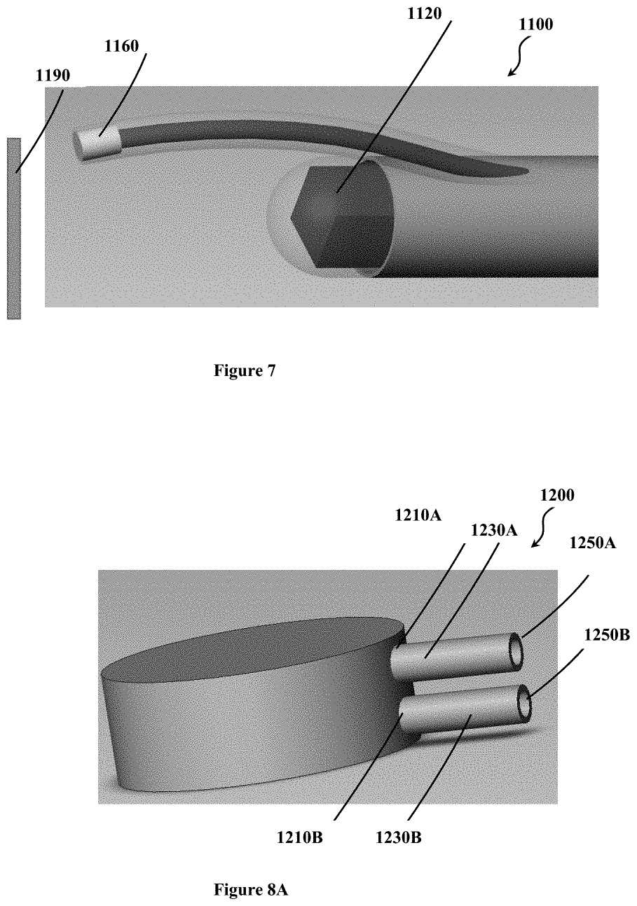

FIG. 7 shows an exemplary representation of an imaging device 1100 according with another embodiment, comprising an illumination element 1160 that is spaced apart from the imaging module 1120. The illumination element 1160 is positioned in close proximity to the target tissue 1190 as compared to the imaging module and is used to illuminate the target tissue.

Under specific circumstances it is desired to illuminate the object at a sharp angle. This is, for example the case with tissue surface when surface details are of interest or when elimination of specular reflection dictates the maximal illumination angle. The current Invention shows that Illumination elements attached to Balloon compartments walls may provide more flexibility in illumination angles and position. A closer-to-object illumination can also reduce the needed illumination power that, as explained earlier, is sometimes an important consideration.

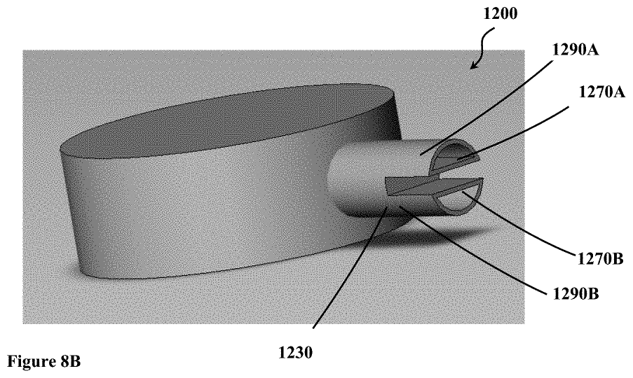

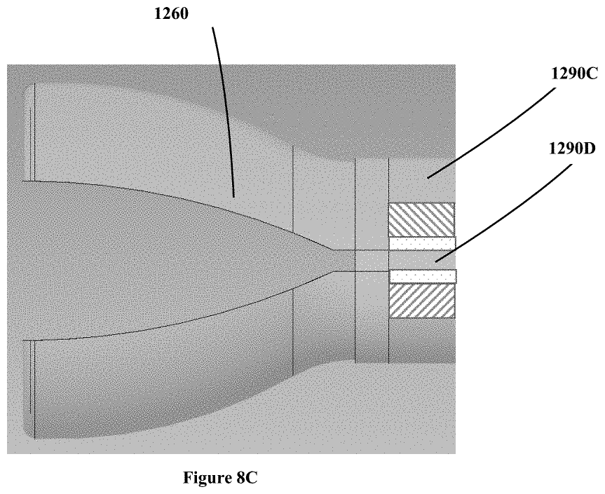

As described herein, the imaging device comprise a pressure system. The pressure system comprises a pump and release valve(s) to inflate and deflate the Balloon. FIGS. 8A to 8C show representative examples of the pressure system. FIG. 8A shows a pump 1200 connected to a imaging device or to delivery member (not shown) through multiple outlets 1210A, 1210B each connected to a different pipeline 1230A, 1230B that may have valves of various shapes, functionalities and in different locations: at the pump outlet 1210A, 1210B inside the pipeline or at the pipeline outlet 1250A, 1250B.

FIG. 8B shows a pump 1200 having a single pipe (tube) 1230C connected to the pump such that the single pipe can split into multiple pipelines 1290A, 1290B, having valves inside of the pipe (not shown) or at the pipe's outlet 1270A, 1270B.

FIG. 8C is a representative embodiment accordingly the inflation is accompanied by an outflow of gas or liquid to clear out the medium in the space between the camera and the captured tissue. Separate pipelines 1290C, 1290D may control the fluid supply to different compartments. The space between the pipes and the shaft wall may also serve as a fluid supply venue for, e.g., flowing fluids through the outlets 1260 onto the line of sight area in-between the camera (not shown) and the target (not shown). The same space may host the power supply and data carrying wires.

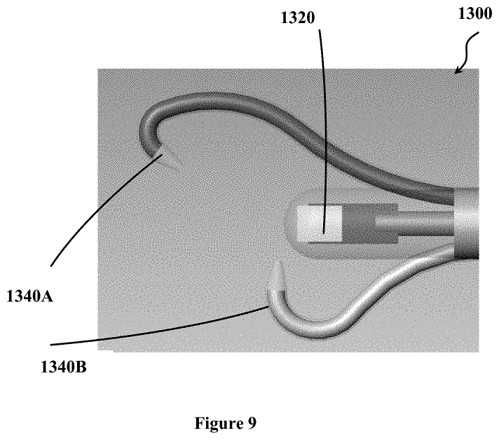

The present disclosure also encompasses the use of at least one support element such as a balloon or a compartment thereof as at least one cleaning means. FIG. 9 shows an exemplary embodiment of an imaging device 1300 comprising an imaging module 1320 and at least one cleaning means 1340A, 1340B such as nozzle. Moreover, due to the sharp angle the jet should form relative to the lens plane, any nozzle adds up to the diameter of the camera probe, which is a grave drawback for mini-cameras that are designed to fit into limited and confined cavities. For example and in accordance with some embodiments, the at least one support element such as a balloon or a compartment thereof may be blown (inflated, expanded) by any suitable manner, for example by gas (e.g., air) or by liquid (e.g., water). When sufficient pressure is built up within the support element, a nozzle, with or without a valve, located on the support element wall, will be expanded, e.g. open up, hence, directing the resultant jet onto the lens surface at a pre-designed angle with respect to it.

Since there may be several nozzle carrying compartments, jets may hit the lens surface from numerous directions, resulting in a better cleaning process.

1. An imaging device comprising an imaging module and at least one support element, wherein the support element or at least a part thereof is configured to undergo inflation in response to a change in pressure.

2. The imaging device according to claim 1, wherein the imaging module is at least one of a camera or an optical fiber and wherein the imaging module is configured to be exposed during image acquisition.

3-4. (canceled)

5. The imaging device according to claim 1, configured to allow a direct transmission of light from a tissue to the imaging module.

6. The imaging device according to claim 1, wherein the support element or any part thereof is any one of (a) configured to undergo a change between a deflated configuration to an inflated configuration and vice-versa in response to a change in pressure, optionally wherein when the support element is in an inflated configuration, the imaging module is exposed; (b) configured to allow at least one of (i) flow of gas and/or fluids and/or air to an imaged tissue, (ii) flow of gas and/or fluids through an imaged tissue and surroundings, and (iii) drainage or gas/fluid supply from and to the tissue; (c) is a membrane or a balloon; or (d) is or comprises an elastic material.

7-10. (canceled)

11. The imaging device according to claim 1, comprising at least one illumination means, optionally comprising at least one selected from the group consisting of a light-emitting diode (LED), a laser, and an optical fiber.

12. (canceled)

13. The imaging device according to claim 1, comprising a delivery member, optionally the delivery member being in a form of a shaft, handle or catheter and the delivery member is configured to undergo association and/or dissociation.

14-15. (canceled)

16. The imaging device according to claim 1, comprising a pressure system coupled with the at least one support element.

17. The imaging device according to claim 1, comprising a tube network configured to allow transfer of fluid and/or gas and/or air from the pressure system to the at least one support element.

18-21. (canceled)

22. A method of imaging a body cavity in a subject, the method comprising applying an imaging device to a subject in need thereof and monitoring a signal from at least one body cavity in the subject, wherein the imaging device comprises an imaging module and at least one support element, wherein the support element or any part thereof is configured to undergo inflation in response to a change in pressure.

23-24. (canceled)

25. The method according to claim 22, wherein the body cavity is at least one of heart, lungs, liver, kidney, stomach and intestines, thymus, pancreas, skin, bone, bone marrow, vascular system, lymph, lymph node, uterus, bladder, fallopian tubes, a joint or ovaries.

26. (canceled)

27. The method according to claim 22, comprising introducing the imaging device into the body cavity such that the support element is in a deflated configuration covering the imaging module.

28. The method according to claim 22, comprising allowing the at least one support element to undergo inflation.

29. The method according to claim 22, comprising applying pressure such that the at least one support element is inflated along a longitudinal optical axis until it is in contact with the tissue.

30. (canceled)

31. The method according to claim 29, comprising inflating the support element perpendicular to a longitudinal optical axis until it is in contact with the tissue.

32. The method according to claim 22, comprising allowing the at least one support element to be inflated by using gas or liquid.

33. The method of claim 22, wherein the imaging device is configured to allow a direct transmission of light from a tissue to the imaging module.

34. The method of claim 22, wherein the support element or any part thereof is any one of (a) configured to undergo a change between a deflated configuration to an inflated configuration and vice-versa in response to a change in pressure, optionally wherein when the support element is in an inflated configuration, the imaging module is exposed; (b) configured to allow at least one of (i) flow of gas and/or fluids and/or air to an imaged tissue, (ii) flow of gas and/or fluids through an imaged tissue and surroundings, and (iii) drainage or gas/fluid supply from and to the tissue; (c) is a membrane or a balloon; or (d) is or comprises an elastic material.

35. The method of claim 22, wherein the imaging device comprises a delivery member, optionally the delivery member being in a form of a shaft, handle, or catheter, wherein the delivery member is configured to undergo association and/or dissociation.

36. The method of claim 22, wherein the signal is indicative of the condition or disease, thereby diagnosing a condition or a disease in the subject.

37. The method according to claim 36, wherein the condition or the disease is associated with uterus (endometrium) status.
